(19) United States
(12) Patent Application Publication
(10) Pub. No.: US 2018/0105806 A1
(43) Pub. Date: Apr. 19, 2018

(54) METHOD FOR RNA-GUIDED ENDONUCLEASE-BASED DNA ASSEMBLY

(71) Applicant: Massachusetts Institute of Technology, Cambridge, MA (US)

(72) Inventors: Christopher Voigt, Belmont, MA (US); Alec Andrew Nielsen, Cambridge, MA (US)

(73) Assignee: Massachusetts Institute of Technology, Cambridge, MA (US)

(21) Appl. No.: 15/696,246

(22) Filed: Sep. 6, 2017

Related U.S. Application Data
(60) Provisional application No. 62/384,339, filed on Sep. 7, 2016.

(51) Int. Cl. C12N 15/10; C12N 15/11; C12N 9/22; C12N 9/00; C12N 9/12

(52) U.S. Cl. CPC C12N 15/102; C12N 15/11; C12N 9/22; C12N 2310/20; C12Y 605/01001; C12N 9/1247; C12Y 207/07006; C12N 9/93

(57) ABSTRACT

The invention provides a novel approach to facilitate assembly of DNA molecules. This approach utilizes RNA-guided endonucleases, capable of targeting any DNA sequence, which cleave DNA and generate DNA fragments characterized by single stranded overhangs. After annealing of complementary overhangs, DNA fragments are covalently connected, generating a single DNA molecule. In this way, the present invention combines the reliability of classic restriction-ligation techniques, removes all sequence constraints from the desired final DNA molecule, and expands the number of DNA pieces that can be assembled at once.

### RELATED APPLICATION

This application claims the benefit under 35 U.S.C. § 119(e) of U.S. provisional application number 62/384,339, filed Sep. 7, 2016, which is incorporated by reference herein in its entirety.

### GOVERNMENT SUPPORT

This invention was made with Government support under Grant No. N00014-13-1-0074 awarded by the Office of Naval Research, and Grant No. P50 GM098792 awarded by the National Institutes of Health. The Government has certain rights in the invention.

### FIELD

The invention generally relates to methods and kits for in vitro DNA assembly.

### BACKGROUND

Recently, it was found that the RNA-guided endonuclease Cpf1 can cleave double stranded DNA (dsDNA) when targeted to a specific locus with a complementary guide RNA. Unlike previously described CRISPR systems (e.g., Cas9) which make “blunt” cuts to the dsDNA (Jinek et al. Science 337, 816-21 (2012)), Cpf1 generates single stranded overhangs 4-5 nucleotides in length (Zetsche et al. Cell 163, 759-71 (2015)). However, previous applications of Cpf1's enzymatic properties have been limited to in vivo gene editing tools (Fagerlund et al. Genome Biot 16, (2015)).

### SUMMARY

Modern genetic engineering relies on the ability to generate custom genetic constructs by “stitching together” pieces of DNA into larger constructs in a process called DNA assembly. Current approaches for DNA assembly are limited by various sequence-based constraints. Technologies for overcoming the limitation of these approaches are needed.

Described herein are novel approaches to facilitate assembly of DNA fragments. Generally, the two approaches utilized for DNA assembly are homology-directed assembly and restriction-ligation assembly. Homology-directed assembly (e.g., Gibson assembly) is not suitable for most complex genetic layouts. Inadvertent homology (even if minor) in regions of DNA can drive formation of incomplete and incorrect final constructs, and the approach is rarely successful when one seeks to combine more than five DNA fragments. RNA-guided endonuclease assembly, which does not rely on the homology of DNA fragments, is not limited by such few DNA fragments.

Restriction-ligation DNA assembly techniques involve restriction endonuclease enzymes, which can generate precise “sticky ends,” which are overhangs in double stranded DNA molecules, i.e., a 5′ or 3′ terminal single stranded portion of a double stranded DNA molecule. These sticky ends can be annealed to complementary sticky ends and covalently linked with a DNA ligase. A modern variant of restriction-ligation relies on Type IIS restriction enzymes, which cut adjacent to their recognition sites instead of within their recognition sites.

All restriction-ligation DNA assembly methodologies, like homology-directed DNA assembly, constrain the design of DNA synthesis for various reasons. First, the DNA sought to be assembled must be devoid of the restriction enzyme recognition sites (except for the assembly junction regions); otherwise, the restriction enzyme will make additional cuts within the DNA. This is problematic because restriction enzyme recognition sites are typically 6 nucleotides in length and randomly occur approximately once per gene. The presence of these sites cannot be avoided when amplifying genes directly from a genome.

In contrast, RNA-guided endonuclease assembly allows complete freedom in selecting the DNA sequences to be assembled, because the guide RNA can be changed to match any desired sequence or recognition site.

Second, restriction enzymes dissociate from the DNA once cut, and “back-ligation” can occur where the flanking recognition site region is ligated back to the newly generated sticky end, regenerating the original DNA molecule instead of the desired assembly. These back-ligation products can be cleaved by the restriction enzyme again, but their ability to form reduces the overall efficiency of the assembly reaction.

In contrast, RNA-guided endonucleases have very low dissociation rates from DNA. After an RNA-guided endonuclease such as Cpf1 cleaves the DNA to which it is bound, Cpf1 remains bound to the flanking site and sterically blocks DNA ligase from “back-ligating” the original DNA strand. Thus, the DNA part-of-interest is free to diffuse and find a desired sticky-end partner without back-ligation. This drives the reaction more efficiently in the forward direction.

Third, the widely used Type IIS enzymes generate sticky ends with 3-4 nucleotide overhangs. Cpf1 generates longer sticky ends of 4-5 nucleotides (depending on the enzyme variant), and this expands the number of unique assembly junctions, and therefore the number of possible DNA parts that can be assembled.

Finally, DNA assembly using an RNA-guided endonuclease, as described herein, provides a simplicity that is not afforded by other DNA assembly methodologies. Of particular note, in a particular embodiment of the disclosed invention, DNA fragments are generated (e.g., via an RNA-guided endonuclease) and ligated (e.g., via a DNA ligase) in a “one-pot” incubation reaction mixture. Additional intermediate steps (e.g., purification steps, enzyme denaturation steps, and modifications to the DNA fragments prior to ligation) and additional reaction components (e.g., joiner oligonucleotides, exonucleases, or polymerases) for DNA assembly are unnecessary.

In some aspects, the invention relates to a method of RNA-guided endonuclease-based DNA assembly comprising: (a) contacting each of at least two DNA molecules with at least one RNA-guided endonuclease and at least one guide RNA molecule under conditions which allow for the generation of at least one double-strand break on each of the at least two DNA molecules, wherein the at least one double-strand break (i) is localized distal to the nucleotide sequence recognized by the guide RNA molecule, and (ii) generates DNA fragments characterized by a single-stranded overhang on at least one of its ends, wherein the single-stranded overhang is complementary to a single-stranded overhang on at least one other DNA fragment generated from at least one other DNA molecule, and (b) contacting the DNA fragments generated in (a) under conditions which allow for (i) hybridization of overhanging ends and (ii) covalent joining of the hybridized ends.

In other aspect, the invention relates to a kit for RNA-guided endonuclease-based DNA assembly comprising: (a) an RNA-guided endonuclease capable of cleaving DNA to form DNA fragments characterized by at least one single-stranded overhang; (b) a DNA ligase; and (c) a reaction buffer.

These and other aspects of the invention are further described below.

### BRIEF DESCRIPTION OF THE DRAWINGS

The following drawings form part of the present specification and are included to further demonstrate certain aspects of the present disclosure, which can be better understood by reference to one or more of these drawings in combination with the detailed description of specific embodiments presented herein. It is to be understood that the data illustrated in the drawings in no way limit the scope of the disclosure.

FIG. 1. Overview of one embodiment of RNA-guided endonuclease assembly. Input DNA molecules (PCR product or plasmid) are flanked by Cpf1 guide RNA recognition sites (black regions). Upon addition of Cpf1 and the guide RNA, sticky ends are generated which are then annealed to complementary sticky ends on other DNA molecules (each 4 nucleotide sticky end sequence is indicated above the assembly junction). T4 DNA ligase then covalently links these pieces of DNA into a large, final construct. The biochemistry of cutting and ligating the DNA occurs in the same reaction mixture.

FIGS. 2A-2C. Purification of Cpf1 orthologues and demonstration of in vitro DNA cleavage. FIG. 2A shows that three Cpf1 orthologs (AsCpf1, FnCpf1, and LbCpf1) were fused to a 6×His purification tag and a maltose-binding protein domain. FIG. 2B shows that the proteins were purified using Nickel NTA resin, and run on an SDS PAGE protein gel. Bands of the expected size for each protein were present. FIG. C shows that the purified Cpf1 protein was incubated with synthesized guide RNA and a dsDNA template in ligase buffer. Cleavage products of the expected size were produced for all three Cpf1 orthologs.

FIGS. 3A and 3B. Proof-of-concept for RNA-guided endonuclease-based DNA assembly using three Cpf1 orthologs. FIG. 3A shows a schematic of the DNA assembly methodology. FIG. 3B shows a representation of the number of colonies observed upon use of each indicated Cpf1 ortholog that were in excess to those of the negative control.

### DETAILED DESCRIPTION

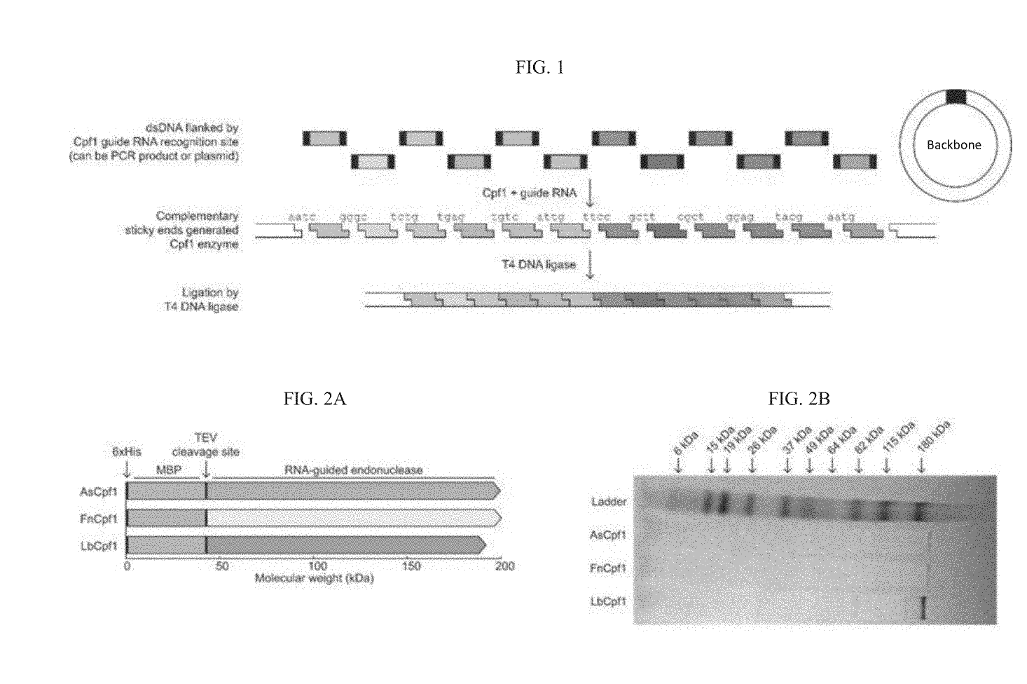

The present invention discloses a novel approach to DNA assembly that includes the reliability of classic restriction-ligation techniques, has fewer or no sequence constraints for generating the desired final DNA molecule, and expands the number of DNA pieces that can be assembled at once (FIG. 1). In a particular embodiment of the disclosed invention, DNA fragments are generated (e.g., via an RNA-guided endonuclease) and ligated (e.g., via a DNA ligase) in a “one-pot” incubation reaction mixture. Additional intermediate steps (e.g., purification steps, enzyme denaturation steps, and modifications to the DNA fragments prior to ligation) and additional reaction components (e.g., joiner oligonucleotides, exonucleases, or polymerases) for DNA assembly are unnecessary.

The term “DNA assembly,” as used herein refers to any process whereby at least two DNA molecules are covalently connected to engineer a single DNA molecule. The term “engineer,” as used herein, refers to a protein molecule, a nucleic acid, complex, substance, or entity that has been designed, produced, prepared, synthesized, and/or manufactured by a human. Accordingly, an engineered product is a product that does not occur in nature.

In some embodiments, the invention discloses a method for RNA-guided endonuclease-based DNA assembly comprising: (a) contacting each of at least two DNA molecules with at least one RNA-guided endonuclease and at least one guide RNA molecule under conditions which allow for the generation of at least one double-strand break on each of the at least two DNA molecules, wherein the at least one double-strand break (i) is localized distal to the nucleotide sequence recognized by the guide RNA molecule, and (ii) generates DNA fragments characterized by a single-stranded overhang on at least one of its ends, wherein the single-stranded overhang is complementary to a single-stranded overhang on at least one other DNA fragment generated from at least one other DNA molecule, and (b) contacting the DNA fragments generated in (a) under conditions which allow for (i) hybridization of overhanging ends and (ii) covalent joining of the hybridized ends.

In some embodiments, steps (a) and (b) are performed or occur in the same reaction mixture and essential simultaneously (e.g., the RNA-guided endonuclease and the ligase are added to the mixture at the same time). In other embodiments steps (a) and (b) are performed or occur sequentially, yet still in the same reaction mixture (e.g., the RNA-guided endonuclease is first added, followed by an incubation time, and then the ligase is added to the same mixture, followed by a second incubation time). Still, in other embodiments, there are intermediate steps between (a) and (b), such as a purification step, such that the two reactions occur in individual reaction mixtures.

While the concentrations of the components utilized in this method (e.g., the RNA-guided endonuclease, the RNA guide molecule, the DNA fragments, and the ligase or the enzyme joining the DNA fragments) may vary, the methods can utilize any effective amount of the components. As such, the contents of the reaction mixtures and the reaction incubation times (nay vary. For example, in some embodiments, the concentration of the guide RNA is about 250 nM and the concentration of the DNA molecules is about 6 nM. it is also recognized by one of ordinary skill in the art that temperature and buffering reagents impact the rate of a reaction. Thus, any incubation length, temperature, or buffering reagent that allows for successful DNA assembly using the components herein can be used according to the methodology disclosed herein.

In some embodiments, at least one of the DNA molecules is a PCR-generated molecule containing at least one region in its nucleotide sequence that is recognized by a guide RNA molecule. In other embodiments, at least one of the DNA molecules is a cloning vector containing at least one region in its nucleotide sequence that is recognized by a guide RNA molecule. In some embodiments, only two DNA fragments are combined, generating a single linear DNA molecule. In other embodiments, only two DNA fragments are combined, generating a single circular DNA molecule. In some embodiments more than two DNA fragments are combined, generating a single linear DNA molecule. In other embodiments, greater than two DNA fragments are combined, generating a single circular DNA molecule.

The term “nuclease,” as used herein, refers to an agent, for example, a protein, capable of cleaving a phosphodiester bond connecting two nucleotide residues in a nucleic acid molecule. A nuclease may be an endonuclease, cleaving a phosphodiester bond within a polynucleotide chain, or an exonuclease, cleaving a phosphodiester bond at the end of the polynucleotide chain. Some nucleases are site-specific nucleases, binding and/or cleaving a specific phosphodiester bond within a specific nucleotide sequence. The terms “recognition sequence,” “recognition site,” “nuclease target site,” or “target site” are used herein to refer to the location where a nuclease interacts with a nucleotide. The recognition sites of many naturally occurring nucleases, for example DNA restriction nucleases, are well known to those of skill in the art. Some endonucleases cut a double-stranded nucleic acid recognition site symmetrically (i.e., cutting both strands at the same position so that the ends comprise base-paired nucleotides), also referred to herein as “blunt ends.” Other endonucleases cut a double-stranded nucleic acid recognition sites asymmetrically (i.e., cutting each strand at a different position so that the ends comprise unpaired nucleotides), also referred to herein as “overhanging ends” or “overhangs” (e.g., as “5′-overhang” or as “3-overhang,” depending on whether the unpaired nucleotide(s) form(s) the 5′ or the 3′ end of the respective DNA strand). Double-stranded DNA molecule ends ending with unpaired nucleotide(s) are also referred to as sticky ends, as they can pair with (“stick to”) other double-stranded DNA molecule ends comprising complementary unpaired nucleotide(s).

In some embodiments, a nuclease is an RNA-guided (i.e., RNA-programmable) nuclease, which is associated with (e.g., binds to) an RNA (e.g., a “guide RNA”, “gRNA,” or “crRNA”) having a sequence that complements a recognition site, thereby providing the sequence specificity of the nuclease. In some embodiments, DNA cleavage by the RNA-guided endonuclease generates a 5′ single stranded overhang that is 4 base pairs in length. In other embodiments, DNA cleavage by the RNA-guided endonuclease generates a 5′ single stranded overhang that is 5 base pairs in length. In some embodiments, the RNA-guided endonuclease cleaves the DNA at the region complimenting the guide RNA. in other embodiments, the RNA-guided endonuclease cleaves the DNA at a location that is distal, proximate, or adjacent to the region complimenting the guide RNA.

In some embodiments, the RNA-guided endonuclease is Cpf1 (see, e.g., Zetsche et al. Cell 163, 759-71 (2015) the entire contents of which are incorporated herein by reference). The terms “Cpf1,” “Cpf1 nuclease,” or “Cpf1 endonuclease” refer to an RNA-guided endonuclease comprising a Cpf1 protein, or a fragment thereof (e.g., a protein comprising an active DNA cleavage domain of Cpf1 and/or an oligonucleotide binding domain of Cpf1). A Cpf1 endonuclease may also be referred to as a CRISPR (clustered regularly interspaced short palindromic repeat)-associated endonuclease or a CRISPR protein. CRISPR is an adaptive immune system that provides protection against mobile genetic elements (viruses, transposable elements and conjugative plasmids). Cpf1-containing CRISPR systems have at least three unique features: (1) Cpf1 -associated CRISPR arrays are processed into crRNAs without the requirement of a trans-acting crRNA; (2) Cpf1-crRNA complexes cleave target DNA proceeded by a short T-rich protospacer-adjacent motif (PAM); and (3) DNA cleavage by Cpf1 generates a double strand break with a 4-5 nucleotide 5′ overhang (Zetsche et al. Cell 163, 759-71 (2015). Cpf1 orthologs have been described in various species, including, but not limited to, Parcubacteria bacterium GWC2011_GWC2_44_17 (BpCpf1), Lachnospiraceae bacterium ND2006 (LbCpf1), Butyrivibrio proteoclasticus (BpCpf1), Peregrinibacteria bacterium GW2011_GWA_33_10 (PeCpf1), Acidarninococcus sp. BV3L6 (AsCPF1), Porphyromonas macacae (PmCpf1), Lachnospiraceae bacterium ND2006 (LbCpf1), Porphyromonas crevioricanis (PcCpf1), Prevotella disiens (PdCpf1), Moraxella bovoculis 237 (MbCpf1), Smithella sp. SC_K08D17 (SsCpf1), Leptospira inadai (LiCpf1), Lachnospiraceae bacterium MA2020 (Lb2Cpf1), Francisella novicida (FnCpf1), Candidatus Methanoplasma termitum (CMtCpf1), and Eubacterium eligens (EeCpf1) (see, e.g., Zetsche et al. Cell 163, 759-71 (2015) the entire contents of which are incorporated herein by reference). In some embodiments, Cpf1 refers to any one of the Cpf1 orthologs described herein, including functional variants or fusion proteins thereof, or other suitable Cpf1 endonucleases and sequences that are apparent to those of ordinary skill in the art. In some embodiments, the term “Cpf1” includes Cpf1 variants which are at least about 70% identical, at least about 80% identical, at least about 90% identical, at least about 95% identical, at least about 98% identical, at least about 99% identical, at least about 99.5% identical, or at least about 99.9% to the native amino acid sequence of a Cpf1 protein. In other embodiments, the term “Cpf1” includes Cpf1 variants which are shorter or longer than the native amino acid sequence of a Cpf1 protein by about 5 amino acids, by about 10 amino acids, by about 15 amino acids, by about 20 amino acids, by about 25 amino acids, by about 30 amino acids, by about 40, amino acids, by about 50 amino acids, by about 75 amino acids, by about 100 amino acids or more, and which retain Cpf1 endonuclease activity. Methods for cloning, generating, and purifying a Cpf1 sequence/protein (or a fragment thereof) are known and apparent to those of skill in the art (see, e.g., Zetsche et al. Cell 163, 759-71 (2015) the entire. contents of which are incorporated herein by reference).

The term “RNA-guided endonuclease” refers to a nuclease that complexes with (e.g., binds or associates with) one or more RNA(s) that is not a target for cleavage. Generally, the bound RNA is referred to as a “guide RNA,” “gRNA” or “crRNA.” Guide RNAs can exist as a complex of two or more RNAs, or as a single RNA molecule. The guide RNA comprises a nucleotide sequence that complements a recognition site, which mediates binding of the nuclease/RNA complex to the recognition site, providing the sequence specificity of the nuclease:RNA complex. Typically, guide RNAs that exist as single RNA species comprise two domains: (1) a “guide” domain that shares homology to a target nucleic acid (e.g., and directs binding of a Cpf1 complex to the target); and (2) a “direct repeat” domain that binds an RNA-guided endonuclease (e.g., a Cpf1 protein). In this way, the sequence and length of the guide RNA may vary depending on the specific recognition site sought and/or the specific RNA-guided endonuclease utilized (see e.g., Zetsche et al. Cell 163, 759-71 (2015) the entire contents of which are incorporated herein by reference). Indeed, all RNA-guided endonuclease are able to bind guide RNAs of various sequences. In some embodiments (e.g., for those utilizing Cpf1 or fusion proteins containing the guide RNA-binding domain thereof), the guide domain of the guide RNA may be 17-25 base pairs in length. Because RNA-guided endonucleases use RNA:DNA hybridization to determine target DNA cleavage sites, these proteins are able to cleave any sequence specified by the guide RNA. In some embodiments, the direct repeat domain may be 16-22 base pairs in length. In some embodiments, the entire length of the guide RNA is 33-47 base pairs in length. In some embodiments, the guide RNA is a universal guide RNA. The term “universal guide RNA” refers to a guide RNA whose sequence is completely independent of the sequence cleaved by the RNA-guided endonuclease. For example, in the context of Cpf1, the guide RNA sequence is completely independent from the sticky end sequence resulting from cleavage. This allows the same guide RNA to be used to generate all possible sticky ends. These guide RNAs may be produced by any method known to one having ordinary skill in the art.

The term “recognition site” refers to a sequence within a nucleic acid molecule that is bound and cleaved by a nuclease. In the context of RNA-guided nucleases, a recognition site typically comprises a nucleotide sequence that is complementary to the guide RNA(s) of the RNA-guided endonuclease, and a protospacer adjacent motif (PAM) at the 3′ end adjacent to the guide RNA-complementary sequence(s). In some embodiments, a recognition site can encompass the particular sequences to which Cpf1 binds. In some embodiments, when the endonuclease is in the form of a fusion protein that contains the binding domain of an RNA guided endonuclease (including, but not limited to, Cas1, Cas3, Cas4, Cas7, Cas9, or Cas10) and the cleavage domain of Cpf1, a recognition site can encompass the particular sequence to which the respective RNA guided endonuclease binds. For the RNA-guided nuclease Cpf1 (or fusion proteins containing the guide RNA-binding domain thereof), the recognition site may be, in some embodiments, 17-25 base pairs in length plus an additional PAM sequence.

PAM sequences vary in length. In some embodiments, the PAM has a length of 3-7 base pairs. In some embodiments, the PAM has a length of 3 base pairs (e.g., NNN, wherein N independently represents any nucleotide). In some embodiments (e.g., where the RNA-guided nuclease has a Cpf1 binding domain), the last nucleotide of a 3 base pair PAM can be any nucleotide, while the other two nucleotides can be either Cor T, but preferably T. In other embodiments (e.g., where the RNA-guided nuclease has a Cas1, Cas3, Cas4, Cas7, Cas9, or Cas10 binding domain), the nucleotide sequence of the PAM depends on the specific Cas protein and its species of origin. Many PAM sequences are known to one having ordinary skill in the art.

The terms “nucleic acid” and “nucleic acid molecule,” as used herein, refer to a compound comprising a nucleobase and an acidic moiety, e.g., a nucleoside, a nucleotide, or a polymer of nucleotides. Typically, polymeric nucleic acids, e.g., nucleic acid molecules comprising three or more nucleotides are linear molecules, in which adjacent nucleotides are linked to each other via a phosphodiester linkage. In some embodiments, “nucleic acid” refers to individual nucleic acid residues (e.g. nucleotides and/or nucleosides). In some embodiments, “nucleic acid” refers to an oligonucleotide chain comprising three or more individual nucleotide residues. As used herein, die terms “oligonucleotide” and “polynucleotide” can be used interchangeably to refer to a polymer of nucleotides (e.g., a string of at least three nucleotides).

In some embodiments, “nucleic acid” encompasses RNA as well as single and/or double-stranded. DNA. Nucleic acids may be naturally occurring, for example, in the context of a genome, a transcript, an mRNA, tRNA, rRNA, siRNA, snRNA, gRNA, a plasmid, cosmid, chromosome, chromatid, or other naturally occurring nucleic acid molecule. On the other hand, a nucleic acid molecule may be a non-naturally occurring molecule, e.g., a recombinant DNA or RNA, an artificial chromosome, an engineered genome, or fragment thereof, or a synthetic DNA, RNA, DNA/RNA hybrid, or including non-naturally occurring nucleotides or nucleosides.

Furthermore, the terms “nucleic acid,” “DNA,” “RNA,” and/or similar terms include nucleic acid analogs, i.e. analogs having other than a phosphodiester backbone. Nucleic acids can he purified from natural sources, produced using recombinant expression systems and optionally purified, chemically synthesized, etc. Where appropriate (e.g., in the case of chemically synthesized molecules), nucleic acids can comprise nucleoside analogs such as analogs having chemically modified bases or sugars, and backbone modifications. These modification may alter a chemical property of the molecules, such as its degradation or binding kinetics. In some embodiments, a nucleic acid is or comprises natural nucleosides (e.g. adenosine, thymidine, guanosine, cytidine, uridine, deoxyadenosine, deoxythymidine, deoxyguanosine, and deoxycytidine); nucleoside analogs (e.g., 2-aminoadenosine, 2-thiothymidine, inosine, pyrrolo-pyrimidine, 3-methyl adenosine, 5-methylcytidine, 2-aminoadenosine, C5-bromouridine, C5-fluorouridine, C5-iodouridine, C5-propynyl-uridine, C5-propynyl-cytidine, C5-methylcytidine, 2-aminoadeno sine, 7-deazaadenosine, 7-deazaguanosine, 8-oxoadenosine, 8-oxoguanosine, O(6)-methylguanine, and 2-thiocytidine); chemically modified bases; biologically modified bases (e.g., methylated bases); intercalated bases; modified sugars (e.g., 2′-fluororibose, ribose, 2′-deoxyribose, arabinose, and hexose); and/or modified phosphate groups (e.g., phosphorothioates and 5′-N-phosphoramidite linkages).

The terms “homology” or “homologous,” as used herein are understood in the art to refer to nucleic acids or polypeptides that are highly related at the level of nucleotide and/or amino acid sequence. Nucleic acids or polypeptides that are homologous to each other are termed “homologues.” Homology between two sequences can be determined by sequence alignment methods known to those of ordinary skill in the art. In accordance with the invention, two sequences are considered to be homologous if they are at least about 50-60% identical, e.g., share identical residues (e.g., amino acid residues) in at least about 50-60% of all residues comprised in one or the other sequence, at least about 70% identical, at least about 80% identical, at least about 90% identical, at least about 95% identical, at least about 98% identical, at least about 99% identical, at least about 99.5% identical, or at least about 99.9% identical, for at least one stretch of at least 20, at least 30, at least 40, at least 50, at least 60, at least 70, at least 80, at least 90, at least 100, at least 120, at least 150, or at least 200 amino acids.

The terms “DNA ligase” or “ligase” refer to a protein that facilitates the formation of a phosphodiester bond between two DNA fragments. Ligases may utilize an ATP-dependent or an ATP-independent reaction mechanism. Prior art has identified and utilized various DNA ligases including, but not limited to, T4 DNA ligase, T3 DNA ligase, T7 DNA ligase, E coli DNA ligase, and Taq DNA ligase. In some embodiments, the DNA ligase is any one of the ligases described herein, or any functional variant or fusion protein thereof retaining ligase activity.

The term “effective amount,” as used herein, refers to an amount of a biologically active agent that is sufficient to elicit a desired biological response. For example, in some embodiments, an effective amount of a nuclease may refer to the amount of the nuclease that is sufficient to induce cleavage of a target site specifically bound and cleaved by the nuclease. As will be appreciated by the skilled artisan, the effective amount of an agent, e.g., a nuclease, a fusion protein, a complex of a protein and a polynucleotide, or a polynucleotide, may vary depending on various factors as, for example, on the desired reaction rate, target site, and components being used.

The present invention also discloses novel kits that provide the components necessary to perform RNA-guided endonuclease assembly. In some embodiments, the kit comprises: (a) an RNA-guided endonuclease capable of cleaving DNA to form DNA fragments characterized by at least one single-stranded overhang; (b) a DNA ligase; and, optionally, (c) a reaction buffer.

In some embodiments, the RNA-guided endonuclease in the kit is Cpf1. Cpf1 orthologs have been described in various species, including, but not limited to, Parcubacteria bacterium GWC2011_C2_44_17 (PbCpf1), Lachnospiraceae bacterium ND2006 (LbCpf1), Butyrivibrio proteoclasticus (BpCpf1), Peregrinibacteria bacterium GW2011_GWA_33_10 (PeCpf1), Acidaminococcus sp. BV3L6 (AsCPF1), Porphyromonas macacae (PmCpf1), Lachnospiraceae bacterium ND2006 (LbCpf1), Porphyromonas crevioricanis (PcCpf1), Prevotella disiens (PdCpf1), Moraxella bovoculis 237 (MbCpf1), Smithella sp. SC_K08D17 (SsCpf1), Leptospira inadai (LiCpf1), Lachnospiraceae bacterium MA2020 (Lb2Cpf1), Francisella novicida (FnCpf1), Candidatus Methwwplasma termitum (CMtCpf1), and Eubacterium eligens (EeCpf1) (see, e.g., Zetsche et al. Cell 163, 759-71 (2015) the entire contents of which are incorporated herein by reference). In some embodiments, the RNA-guided endonuclease in the kit is a variant of a Cpf1 ortholog which is at least about 70% identical, at least about 80% identical, at least about 90% identical, at least about 95% identical, at least about 98% identical, at least about 99% identical, at least about 99.5% identical, or at least about 99.9% to the native sequence of a Cpf1 protein, and which retains Cpf1 endonuclease activity. in other embodiments, the Cpf1 variants are shorter or longer than the native sequence of a Cpf1 protein by about 5 amino acids, by about 10 amino acids, by about 15 amino acids, by about 20 amino acids, by about amino acids, by about 30 amino acids, by about 40 amino acids, by about 50 amino acids, by about 75 amino acids, by about 100 amino acids or more, and retain Cpf1 endonuclease activity. In other embodiments, the RNA-guided endonuclease is a fusion protein that contains the binding domain of an RNA guided endonuclease (including, but not limited to, Cas 1, Cas3, Cas4, Cas7, Cas9, or Cas10) and the cleavage domain of Cpf1 or a variant thereof, and which fusion protein retains Cpf1 endonuclease activity.

In some embodiments, the ligase in the kit is T4 DNA ligase and the reaction buffers (1X) preferably includes 50 mM Tris-HCl, 10 mM MgCl₂, 1 mM ATP, and 10 mM DTT at a pH 7.5 at 25 degrees Celsius. In other embodiments, the ligase is a different known ligase and corresponding buffer composition.

In some embodiments, the kit also comprises one or more guide RNA(s). In some embodiments, the kit also comprises a universal guide RNA. In some embodiment the guide RNA molecule is provided in a desiccated or lyophilized form. In other embodiments the guide RNA is provided in a precipitated form. In other embodiments the guide RNA is provided in a solubilized form. In other embodiments, the kit also comprises reagents sufficient for the production of an RNA guide molecule. Mechanisms for generating guide RNA molecules are known to those having ordinary skill in the art, and include, but are not limited to, using an RNA polymerase to produce an RNA molecule from a DNA molecule (e.g., linear or circular, synthesized or PCR generated) encoding the sequence of at least one guide RNA, wherein an RNA polymerase promoter is localized 5′ to the sequence.

In some embodiments, components of the reaction described herein are provided in cocktail form. For example, in some embodiments, the RNA-guided endonuclease and the DNA ligase are combined in a cocktail form. In other embodiments, the RNA-guided endonuclease and universal guide RNA, and optionally the DNA ligase are combined in a cocktail form.

In some embodiments, the kit also comprises a cloning vector containing at least one sequence recognized by a guide RNA. In some embodiments, the kit also comprises competent cells for use in the cloning of the desired DNA assembly molecule. For example, in some embodiments, the competent cells used are chosen from the list comprising TOP10, OmniMax, PIR1, PIR2, INV αF, IN110, BL21, Mach1, DH10Bac, DH10B, DH12S, DH5α, Stb12, Stb13, and Stb14. XL1-Blue, XL2-Blue, and related strains.

### EXAMPLES

### Methods and Materials

### Purification of Cpf1 Orthologs

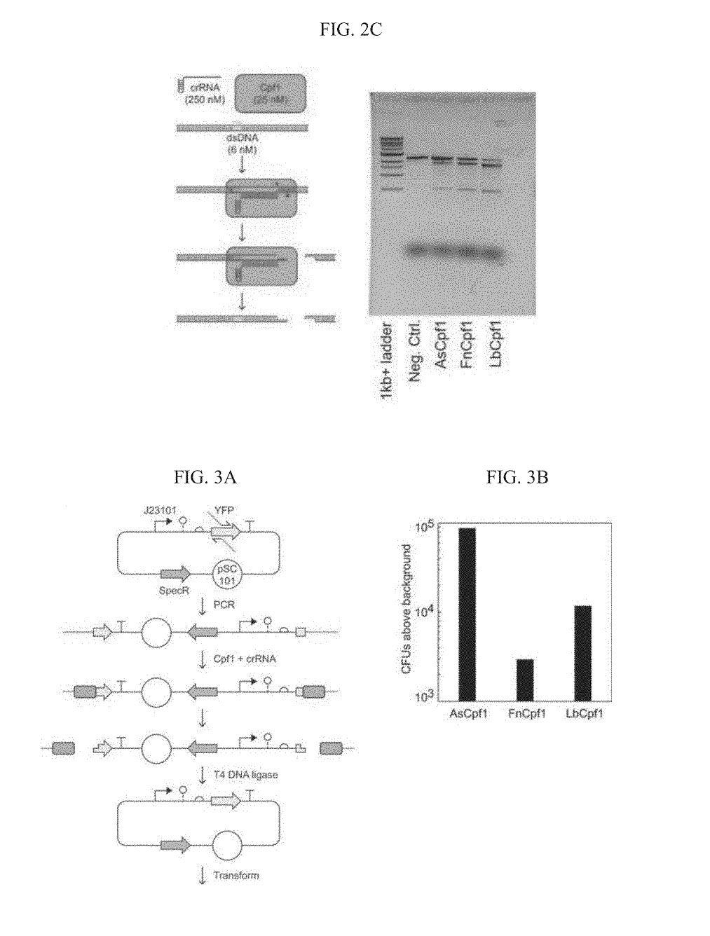

Clones of three 5′-tagged Cpf1 orthologs (AsCpf1, FnCpf1, and LbCpf1) were generated. The sequence of each tag, from 5′ to 3′, consists of a 6×His purification tag, a maltose-binding protein domain, and a TEV cleavage site (FIG. 2A). Each protein ortholog was purified using Nickel NTA resin, and run on an SDS PAGE protein gel. A band at the expected size for each protein was present (FIG. 2B).

### Synthesis of Guide RNAs

Guide RNAs were synthesizedaccording to standard procedures.

### Synthesis of Cpf1-Cleavable DsDNA Fragments

Cpf1-Cleavable dsDNA fragments were synthesized according to standard procedures.

### In Vitro DNA Cleavage and Assembly

Cpf1-cleavable dsDNA fragments were incubated with a Cpf1 protein, its corresponding synthesized guide RNA, NEB T4 DNA ligase (New England Biolabs, Ipswich, MA), and NEB T4 DNA ligase buffer (New England Biolabs) at 37° C. for 2 hours.

### Example 1

### Demonstration of In Vitro DNA Cleavage

Each purified Cpf1 protein ortholog (AsCpf1, FnCpf1, and LbCpf1) was incubated with its corresponding guide RNA (250 nM) and a Cpf1-cleavable dsDNA template (6 nM) in ligase buffer. Cleavage products of the expected size were produced for all three Cpf1 orthologs (FIG. 2C).

### Example 2

### Proof-of-Concept for RNA-Guided Endonuclease-Based DNA Assemboy

Yellow fluorescent protein (YFP)-containing circular plasmids were PCR-amplified to produce linear double stranded DNA fragments using primers that contain the Cpf1 guide RNA annealing sites in their tails. These fragments were then incubated with a purified Cpf1 ortholog, its corresponding guide RNA, NEB T4 DNA ligase, and NEB T4 DNA ligase buffer at 37° C. for 2 hours (FIG. 3A). The reactions were purified and transformed into chemically competent E. coil, and serial dilutions were plated on selective agar media. The next day, the number of colonies for each Cpf1 ortholog were counted. Reactions involving each Cpf1 ortholog successfully produced colony numbers above the background number of colonies from the negative control (FIG. 3B).

### REFERENCES

1. Jinek M., Chylinski K., Fonfara I., Hauer M., Doudna J. A., and Charpentier E., A programmable dual-RNA-guided DNA endonuclease in adaptive bacterial immunity. Science. Aug. 17, 2012; 337(6096):816-21.
2. Zetsche B., Gootenberg J. S., Abudayyeh O. O., Slaymaker I. M., Makarova K. S., Essletzbichler P., Volz S. E., Joung J., Oost J., Regev A., Koonin E. V., and Zhang F., Cpf1 is a single RNA-guided endonuclease of a class 2 CRISPR-Cas system. Cell. Oct. 22, 2015; 163(3):759-71.
3. Fagerlund R. D., Staals R. H., and Fineran P. C., The Cpf1 CRISPR-Cas protein expands genome-editing tools. Genome Biol. Nov. 17, 2015; 16:251.

### OTHER EMBODIMENTS

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present disclosure, and without departing from the spirit and scope thereof, can make various changes and modifications of the disclosure to adapt it to various usages and conditions. Thus, other embodiments are also within the claims.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the. inventive teachings is/are used. Those skilled in the art will recognize, or be able to ascertain using no more than routine experimentation, many equivalents to the specific inventive embodiments described herein. it is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A) in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” contatntn©, “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03. It should be appreciated that embodiments described in this document using an open-ended transitional phrase (e.g., “comprising”) are also contemplated, in alternative embodiments, as “consisting of” and “consisting essentially of” the feature described by the open-ended transitional phrase. For example, if the disclosure describes “a composition comprising A and B”, the disclosure also contemplates the alternative embodiments “a composition consisting of A and B” and “a composition consisting essentially of A and B”.

1. A method for RNA-guided endonuclease-based DNA assembly comprising:
(a) contacting each of at least two DNA molecules with at least one RNA-guided endonuclease and at least one guide RNA molecule under conditions which allow for the generation of at least one double-strand break on each of the at least two DNA molecules, wherein the at least one double-strand break
(i) is localized within five base pairs to the nucleotide sequence recognized by the guide RNA molecule, and
(ii) generates DNA fragments characterized by a single-stranded overhang on at least one of its ends, wherein the single-stranded overhang is complementary to a single-stranded overhang on at least one other DNA fragment generated from at least one other DNA molecule, and
(b) contacting the DNA fragments generated in (a) under conditions which allow for
(i) hybridization of overhanging ends and (ii) covalent joining of the hybridized ends.

2. The method of claim 1, wherein steps (a) and (b) occur in the same mixture.

3. The method of claim 1, wherein the single-strand overhangs are 4-5 nucleotides in length.

4. The method of claim 1, wherein at least one of the DNA molecules is a PCR-generated molecule containing at least one nucleotide sequence recognized by a guide RNA molecule.

5. The method of claim 1, wherein at least one

6. The method of claim 1, wherein the guide RNA molecule is a universal guide RNA molecule.

7. A kit for RNA-guided endonuclease-based DNA assembly comprising:
(a) an RNA-guided endonuclease capable of cleaving DNA in the presence of a guide RNA to form DNA fragments characterized by at least one single-stranded overhang,
(b) a DNA ligase, and
(c) optionally, a reaction buffer.

8. The kit of claim 7, wherein the RNA-guided endonuclease and the DNA ligase are combined in a cocktail form.

9. The kit of claim 7, additionally comprising a DNA molecule encoding for the sequence of at least one universal guide RNA, wherein an RNA polymerase promoter is localized 5′ to the sequence.

10. The kit of claim 7, additionally comprising an RNA polymerase.

11. The kit of claim 7, additionally comprising a universal guide RNA.

12. The kit of claim 7, wherein the Cpf1 endonuclease, universal guide RNA, or DNA ligase are combined in a cocktail form.

13. The kit of claim 7, additionally comprising a cloning vector containing at least one sequence recognized by the guide RNA.

14. The kit of claim 7, additionally comprising competent cells for use in cloning a DNA molecule assembled using the kit.
